(19) United States
(12) Patent Application Publication
(10) Pub. No.: US 2015/0290388 A1
(43) Pub. Date: Oct. 15, 2015

(54) CATHETER FOR VASCULAR IMAGING, METHOD OF IMAGING, AND METHOD FOR INTERVENTIONAL PROCEDURE USING THE CATHETER

(71) Applicant: Subramaniam C. KRISHNAN, Sacramento, CA (US)

(72) Inventor: Subramaniam C. KRISHNAN, Sacramento, CA (US)

(21) Appl. No.: 14/748,978

(22) Filed: Jun. 24, 2015

Related U.S. Application Data
(63) Continuation of application No. PCT/IB2013/061429, filed on Dec. 31, 2013.
(60) Provisional application No. 61/747,679, filed on Dec. 31, 2012.

(51) Int. Cl. A61M 5/00; A61B 18/00; A61M 25/09; A61B 5/055; A61B 17/12

(52) U.S. Cl. CPC A61M 5/007; A61B 5/055; A61B 17/12136; A61B 17/12109; A61B 17/1204; A61M 25/09; A61B 18/00; A61B 2017/12004; A61B 2018/0022; A61B 2018/00577

(57) ABSTRACT

A catheter for cardiac imaging, a method of imaging and a method of conducting an interventional procedure using the catheter is disclosed, where the catheter includes a tube with one or more openings for releasing an intravenous contrast agent, and an inflatable cuff holding a portion of the tube. The inflatable cuff includes a dorsal surface, and a ventral surface, where the ventral surface has a hollow compartment, an inner edge and an outer edge. The inflatable cuff functions as a seal to prevent an extrusion of an intravenous contrast agent supplied through one or more openings, in a dorsal direction.

### CROSS-REFERENCE TO RELATED APPLICATIONS

This application is a continuation of PCT International Patent Application No. PCT/IB2013/061429 filed Dec. 31, 2013, which claims benefit of priority to U.S. Provisional Patent Application No. 61/747,679 filed Dec. 31, 2012, the content of each of which is incorporated herein by reference in its entirety.

### FIELD OF THE INVENTION

The invention relates generally to a catheter and more specifically to a catheter for vascular imaging, useful in vascular imaging and cardiovascular interventional procedure.

### BACKGROUND OF THE INVENTION

Catheters are medical devices in the form of a long narrow tube that can be inserted in the body cavity, duct, or vessel to image a bodily internal region, to treat diseases or to perform a surgical or invasive procedure. Catheters are used in cardiovascular, urological, gastrointestinal, neurovascular, ophthalmologic and other applications. The commonest cardiac procedures performed with catheters are coronary angiography. Conventional catheters to perform angiography or venography involve a hollow tube where the tip is inserted directly inside the vessel of interest and the contrast agent is then injected.

Coronary angiography or venography of a vessel is an invasive procedure done by inserting a catheter into a blood vessel in the arm or leg of a patient and guiding the catheter under X-ray guidance directly into the vessel of interest. The main purpose of angiography is to detect narrowing or occlusive disease. Based on the results of angiography, interventional procedures include balloon angioplasty, brachytherapy, atherectomy, rotoblation, cutting balloon, and stent placements may be performed. A venogram is performed to understand the course of vessel. Coronary venography is often performed prior to inserting a pacing lead to perform pacing of the left ventricle. Yet another need for performing angiography or venography is during catheter ablation procedure to treat abnormal heart rhythms. Blood vessels or accompanying structures may be immediately adjacent to the ablation catheter. Continuing with the ablation procedure may result in damage to the blood vessel or the structure for example a nerve accompanying the blood vessel. Phrenic nerve is one such structure in the heart that can get damaged with ablations performed for treatment of Atrial Fibrillation and other arrhythmias (heart rhythm disorder). This is especially true with cryoablation performed with balloons in the right pulmonary veins, in the left atrium of the heart. Prior studies show that the incidence of right phrenic nerve injury can be as high as 10% in these procedures.

During the imaging and treatment procedures there are certain regions in the heart or cardiovascular system that are not easy to navigate or reach. One anatomical factor that can prevent a direct canulation of the catheter into the ostium of the vessel of interest is the size or caliber of the vessel of interest or the angle that it makes as it branches from the larger vessel.

It is therefore important to detect the phrenic nerve properly and then ensure that it is protected during any interventional procedure being performed in its proximity. However, this poses difficulty, as the angle that the right pericardiophrenic vein (this vein accompanies the phrenic nerve) with the superior vena cava or SVC makes selective cannulation of right pericardiophrenic vein with an angiography catheter very difficult. Due to its unique positioning, venography (x-ray of the veins) of right pericardiophrenic vein for real time visualization is difficult with the existing imaging techniques. Venography and radiological imaging of the right pericardiophrenic vein is important since it can detect and outline the course of the right phrenic nerve, which would be useful in planning any interventional treatment procedure such as an ablation in the vicinity of the right phrenic nerve.

### BRIEF SUMMARY OF THE INVENTION

There are certain anatomical structures in the heart which overlap or lie in close proximity to the vessels of interest where catheter for imaging a region of interest needs to be deployed. There is a need to access the vessels of interest at the same time not to cause any injury to other anatomical structures in the imaging or in the interventional treatment procedures. Inserting the angiography catheter is easier when the long axis of the vessel of interest (that arises from the larger vessel) is perpendicular to the larger vessel. If the vessel of interest arises at a very steep acute or obtuse angle from the larger vessel, it can be difficult to directly cannulate this vessel with the conventional angiography catheter. Under these circumstances, the availability of a catheter of the invention that allows for nonselective contrast can be very important.

The principal object of the invention is to provide a novel catheter with an inflatable cuff that serves to visualize the vessel of interest by injecting contrast in a nonselective manner and protect the anatomic structures in proximity of the vessel of interest, and also to provide a seal for an intravenous contrast agent that is used for imaging the region of interest.

Another object of the invention is to provide a method of cardiac imaging using the catheter of the invention.

Yet another object of the invention is to provide a method for interventional treatment using the catheter of the invention. The interventions include locating areas of trauma to arteries and veins and using the catheters system to achieve hemostasis using an endovascular approach.

In one aspect of the invention, a catheter for vascular imaging via non-selective contrast injection is disclosed, where the catheter includes a tube with one or more openings for releasing a radio-opaque contrast agent, and an inflatable cuff holding a portion of the tube. The inflatable cuff includes a dorsal surface, and a ventral surface, where the ventral surface has a hollow compartment, an inner edge and an outer edge. The inflatable cuff functions as a seal to prevent an extrusion of an intravenous contrast agent supplied through one or more openings. Other enhancements for the inflatable cuff are also described.

In another aspect of the invention a method of vascular imaging using the catheter as described herein above is disclosed. The method includes steps for deploying the catheter adjacent to a vessel of interest using a guidewire; rotating the catheter such that the ventral surface of the inflatable cuff faces towards the ostium of the vessel of interest; inflating the inflatable cuff to expand as a balloon to envelop a desired location of the ostium of the vessel of interest; deflecting the catheter towards a vessel wall of the vessel of interest, for example a lateral wall of superior vena cava; applying suction force through cuff openings to create a seal between the vessel wall and the inflatable cuff; injecting an intravenous contrast agent into the tube of the catheter; and imaging the vessel of interest by detecting the intravenous contrast agent in the vessel of interest. The inflatable cuff functions as a seal to prevent an extrusion or leakage of the intravenous contrast agent into the lumen of a larger vessel and thus to force the contrast agent into the lumen of the branching vessel of interest.

In another aspect, method for doing interventional treatments using the catheter of the invention is also disclosed. These interventions include but are not limited to using the catheter of this invention to deliver wires, balloons and stents to the target vessel to perform percutaneous coronary interventions. It is also anticipated that this catheter of the invention can be used in situations where there is trauma to blood vessels due to a tear. One example of such a scenario is a tear in a thoracic vein such as the superior vena cava that can occur during extraction of chronic pacemaker and defibrillator leads.

### BRIEF DESCRIPTION OF THE SEVERAL VIEWS OF THE DRAWINGS

These and other features, aspects, and advantages of the present invention will become better understood when the following detailed description is read with reference to the accompanying drawings in which like reference numerals represent corresponding parts throughout the drawings, wherein:

FIG. 1 is a diagrammatic representation of the heart; Also seen are the right and left pericardiophrenic veins. These veins along with the pericardiophrenic arteries accompany the right and left phrenic nerves and form the pericardiophrenic bundles. Imaging the vascular structures will provide a location of the nerves as well.

FIG. 2 is a sectional view of the catheter showing the ventral surface of the inflatable cuff, according to one embodiment of the invention;

FIG. 3 is a diagrammatic view of the catheter according to another embodiment of the invention wherein the ventral surface has a concavity with a central hollow and the cuff on the ventral surface with a concavity that has openings to allow for suction and to thus create a watertight seal;

FIG. 4 is a diagrammatic representation of the catheter according to yet another embodiment where the ventral surface is made from a material impervious to an intravenous contrast agent;

FIG. 5 is diagrammatic view of the catheter integrating an ablation balloon and the inflatable cuff, according to yet another embodiment of the invention;

FIG. 6 is a diagrammatic view showing the catheter of the present embodiments deployed in the heart proximal to pericardiophrenic vein (vessel of interest) through one exemplary approach;

FIG. 7 is a diagrammatic view showing the catheter of the present embodiments deployed in the heart proximal to pericardiophrenic vein through another exemplary approach;

FIG. 8 is diagrammatic view showing the placement of the catheter of the invention with the guide wire proximal to another vessel of interest, in this instance an aortocoronary vein graft;

FIG. 9 is a flowchart representation of a method of cardiac imaging using the catheter of the invention;

FIG. 10 is a diagrammatic representation showing the deployment of the catheter of the invention along with an ablation balloon during an interventional treatment procedure;

FIG. 11 is a diagrammatic representation showing the deployment of the catheter of the invention and the ablation balloon on same guidewire;

FIG. 12 is a diagrammatic representation showing the deployment of two catheters of the invention at a site of trauma having a vessel tear;

FIG. 13 is a diagrammatic representation showing the deployment of a catheter of the invention to locate a site of trauma having a vessel tear and placing a wire at the site; and

FIG. 14 is a diagrammatic representation showing the deployment of a catheter at the site of trauma where an expandable element is advanced over the wire of FIG. 13.

### DETAILED DESCRIPTION OF THE INVENTION

As used herein and in the claims, the singular forms “a,” “an,” and “the” include the plural reference unless the context clearly indicates otherwise.

The invention described herein has applications in vascular imaging and in certain invasive cardiac procedures. The application contains reference to certain anatomical regions and features of the heart, and these are well understood by those skilled in the art. Definitions for some terms has been provided herein, and for others the normal medical definition as would be understood by one in this field applies. Ostium as referred herein is an opening of coronary arteries at root of aorta. Pericardiophrenic bundle as referred herein includes phernic nerve, pericardiophrenic artery and pericardiophrenic vein.

Aspects of the invention are also useful in non-selective angiography, where the contrast agent needs to be injected into a vein. It will be understood by those skilled in the art that in cases where the target vessel, referred herein as a vessel of interest arises from a larger vessel at an extremely acute or obtuse angle, direct or selective angiography or venography is not possible. Similarly, where the catheter diameter is larger that of target vessel, there also selective angiography or venography is not possible. In these scenarios non-selective angiography using the catheter of the invention is able to overcome these constraints. Further, due to the superimpositions of other structures around this region as discussed herein above, it is important that the catheter for vascular imaging or for interventional procedures does not damage other structures around the cardiac region and also the contrast agent reaches the desired location. Both these objectives are achieved by the catheter of the invention.

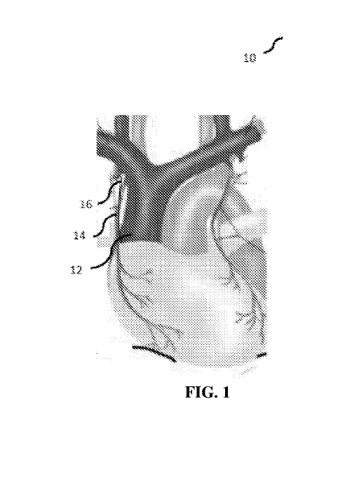

FIG. 1 is a diagrammatic representation of the heart 10 showing different regions that are referred to in the description herein in relation with different embodiments of the invention. Referral numeral 12 indicates superior vena cava or SVC (vein that carries deoxygenated blood from the upper half of the body to the heart's right atrium), 14 indicates right pericardiophrenic vein and 16 indicates the phrenic nerve. One skilled in the art would be aware of the anatomy of the right pericardiophrenic bundle in the heart and the proximity of the phrenic nerve to pericardiophrenic vein in this bundle.

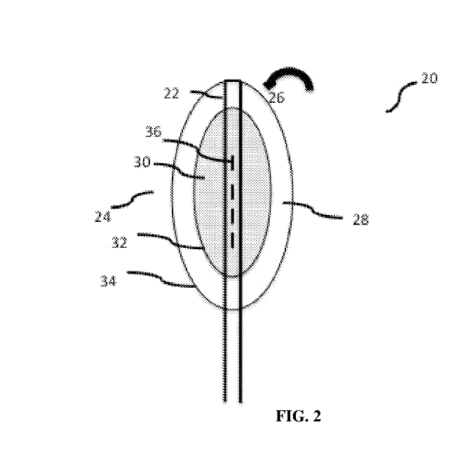

FIG. 2 is a sectional view of an exemplary embodiment of the catheter 20 according to one embodiment of the invention. As is seen in FIG. 2, the catheter 20 includes a tube 22, with one or more openings 36 for releasing a radio-opaque contrast agent and additionally, and advantageously the catheter 20 includes an inflatable cuff 24 holding a portion of the tube. The inflatable cuff has a dorsal surface 26 (shown by block arrow, indicating the reverse side of the drawing), and a ventral surface 28. The ventral surface 28 has a concavity with a central hollow compartment or a depression 30 and an inner edge 32 and an outer edge 34 of the ventral surface 28. This inflatable cuff functions as a seal or a plug to prevent an extrusion in a dorsal direction of an intravenous contrast agent supplied through one or more openings of the catheter tube. In some embodiments, the inflatable cuff can be expanded as an inflatable balloon useful in certain cardiac procedures. Use of intravenous contrast agents for imaging purposes is well known and a number of substances have been used as positive contrast agents such as silver, bismuth, caesium, gadolinium, thorium, tin, zirconium, tantalum, tungsten and lanthanide compounds. Iodine-based positive contrast agents have also been used.

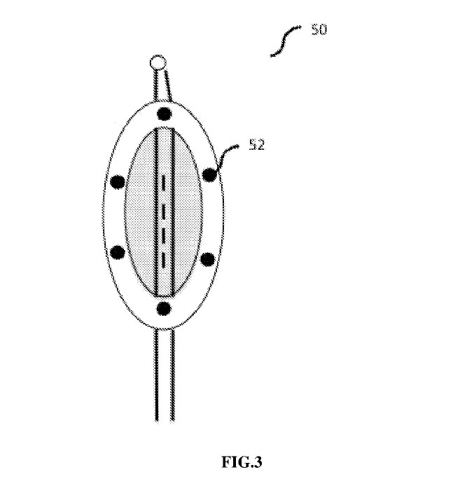

FIG. 3 is a diagrammatic (sectional) view of another embodiment 50 of the catheter of the invention where the inflatable cuff is provided with cuff openings 52 between the inner edge and outer edge on the ventral surface for implementing a vacuum suction mechanism along the inflatable cuff. This creates a “water tight” seal and improves the adherence of the inflatable cuff to the vessel wall. In another embodiment, the catheter on the ventral surface includes magnets such as electromagnets placed between the inner edge and outer edge of the ventral surface of the inflatable cuff. The electromagnets are useful for proper positioning of the inflatable cuff inside a cardiac region.

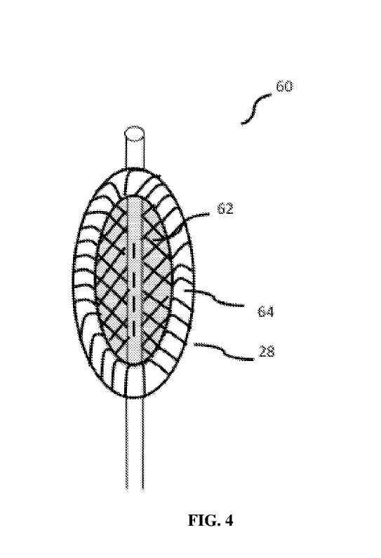

FIG. 4 is a diagrammatic (sectional) view of another embodiment 60 of the of the catheter of the invention where the ventral surface 28 includes or is made from a material 62 that is impermeable to the intravenous contrast agent. Some non-limiting examples of the material include a fabric material or a polymer material. Further as another exemplary embodiment or within the embodiment of FIG. 4, the outer edge and the inner edge of the ventral surface 28 may include a compressible metallic frame 64. In one example, the compressible metallic frame is made from nitinol.

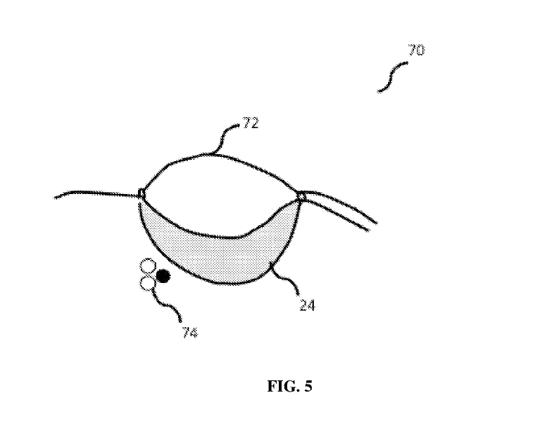

FIG. 5 is a diagrammatic representation of yet another implementation 70 of the catheter of the invention showing an ablation balloon 72 integrated with the inflatable cuff 24 that is expanded as an inflatable balloon for certain cardiac interventional procedures as explained in more detail herein below. During such procedures, when the catheter is placed proximal to the pericardiophrenic bundle 74, upon inflation with a gas or another material the inflatable balloon serves as a thermal insulator, preventing damage to crucial structures such as the phrenic nerve.

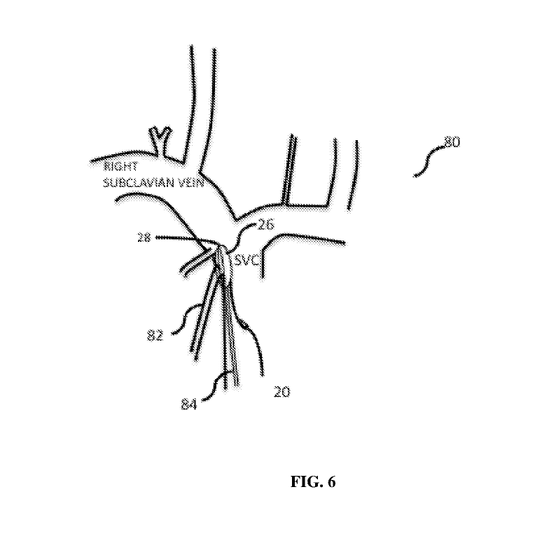

FIG. 6 is a diagrammatic representation 80 showing the catheter 20 of FIG. 2 or other embodiments in FIG. 3-5 of the invention placed inside the heart in proximity to the right pericardiophrenic vein 82. The ventral surface 28 of the inflatable cuff faces the right pericardiophrenic vein 82, while the dorsal surface 26 provides a closed encapsulating cover to prevent the contrast agent to flow outwards. The inflatable cuff therefore surrounds or occludes the area adjacent to the ostium of the right pericardiophrenic vein. Reference numeral 84 indicates use of pull wire technology to deflect a shaft of the catheter towards the ostium of the right pericardiophrenic vein 82 for positioning the inflatable cuff appropriately for injecting the contrast agent into the right pericardiophrenic vein 82. As shown in this drawing, the catheter of the invention is deployed at the junction between right subclavian vein and SVC over a wire. In an exemplary implementation, the wire is rotated so that ventral surface 28 faces rightwards and laterally towards the right pericardiophrenic vein 82. The balloon cuff is then deployed to envelop the ostium of the right pericardiophrenic vein 82. The inflatable cuff after deployment forms a tight seal and the contrast agent is prevented from being extruded in the dorsal direction, and enters the lumen of the target vessel i.e. the right pericardiophrenic vein 82 in this case.

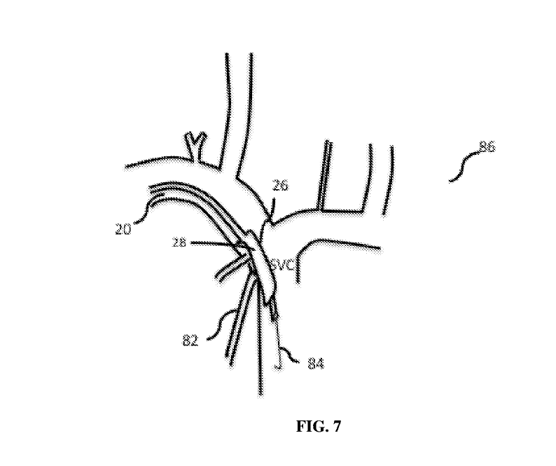

FIG. 7 is a diagrammatic representation 86 showing the catheter 20 inserted into the cardiac region to reach the right pericardiophrenic vein 82 through another approach, via the subclavian or axillary vein. In this approach, it is possible to direct a wire 84 into the right pericardiophrenic vein 82 guided by contrast imaging of the vein. The inflatable cuff advantageously prevents the wire from going down the SVC.

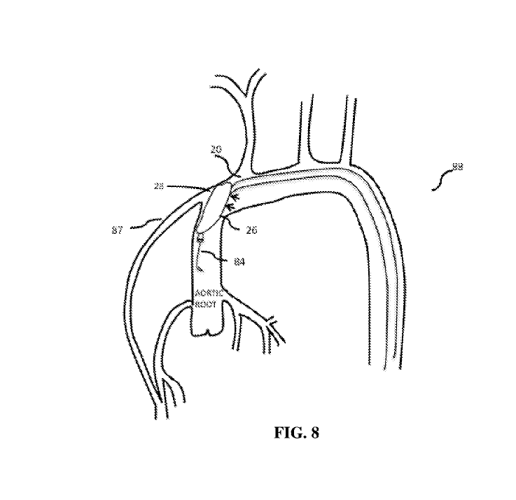

FIG. 8 is a diagrammatic representation 88 showing catheter 20 for imaging aortocoronary vein grafts such as a saphenous vein graft 87 using a similar approach as described herein above, where the ventral surface 28 faces the vein graft and dorsal surface 26 forms a seal.

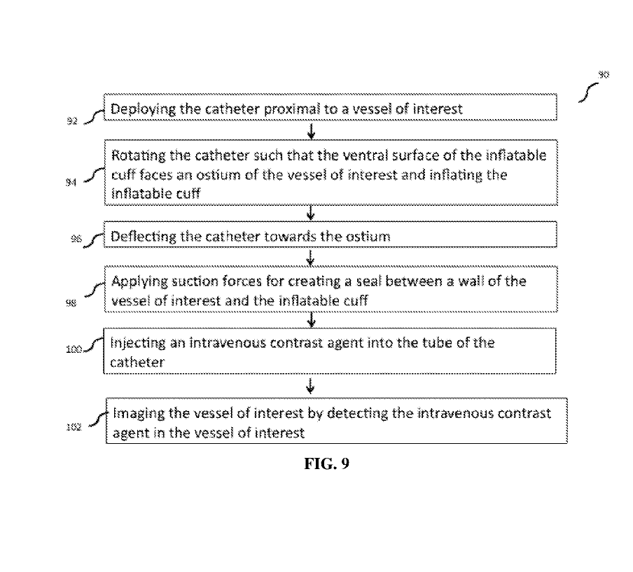

An exemplary method for imaging a cardiac vessel of interest or region of interest using the catheter (with the inflatable cuff) of the invention is shown as flowchart 90 in FIG. 9. The catheter in an exemplary implementation may be inserted inside a larger vessel from which the target vessel of interest arises as shown in FIG. 7-9. At step 92 the catheter is deployed adjacent to the vessel of interest using for example, a guidewire proximal to the vessel of interest. The deployment is done through subclavian vein into the pericardiophrenic vein in one example. The catheter is deployed in another exemplary procedure at the junction between the right subclavian vein and the SVC. It would be appreciated by those skilled in the art that the presence of the inflatable cuff prevents the guidewire from going down the SVC. The catheter is then rotated as shown at step 94 so that the ventral surface (with hollow) of the inflatable cuff faces ostium of the target vessel or vessel of interest and the cuff is inflated with gas or liquid. Using pull wire technology or with other mechanisms, the ventral surface is bent (turned or pressed) towards the ostium of the vessel of interest at step 96. The deflection of the catheter in another implementation is achieved by incorporating electromagnets of the ventral surface of the inflatable cuff of the catheter. Suction forces are applied through the openings in the inflatable cuff to create a “watertight seal” between the vessel wall and the inflatable cuff at step 98. Once the inflatable cuff is deployed in desired position as mentioned herein, the intravenous contrast agent is injected at step 100 into the vessel of interest by injecting contrast agent into the vessel without directly canulating the vessel. As mentioned herein above due to the presence of the inflatable cuff that functions as a seal, the intravenous contrast agent is prevented to flow out in the dorsal direction, it enters the lumen of the vessel of interest as is desired and thus greatly enhancing the imaging of the vessel at step 102.

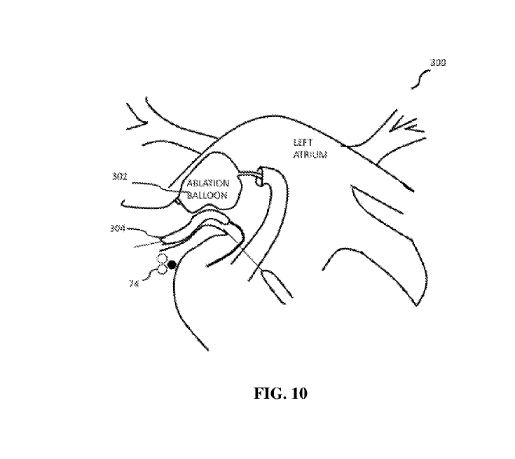

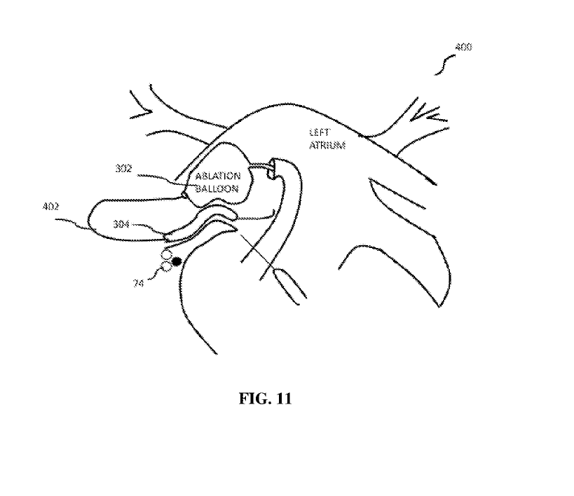

In yet another implementation 300 as shown in FIG. 10, if the imaging catheter reveals the phrenic nerve to be adjacent to the ablation balloon 302, a separate shielding balloon 304 is placed adjacent to the phrenic nerve to prevent any ablation-induced damage to the cardiac structure, where the shielding balloon is the inflatable cuff of the catheter of the invention and is inflated with a material (gas) that is likely to not conduct heat or cold. The pericardiophrenic bundle 74 is also shown. In another exemplary implementation 400 as shown in FIG. 11, the shielding balloon 304 is advanced over the same guidewire 402 that is used to advance the ablation balloon 302. In yet another alternate embodiment, the ablation balloon has the inflatable cuff in its outer segment as shown in FIG. 6 as an integrated catheter. Upon inflation with a gas or another material the inflatable balloon serves as a thermal insulator, preventing damage to crucial structures such as the phrenic nerve.

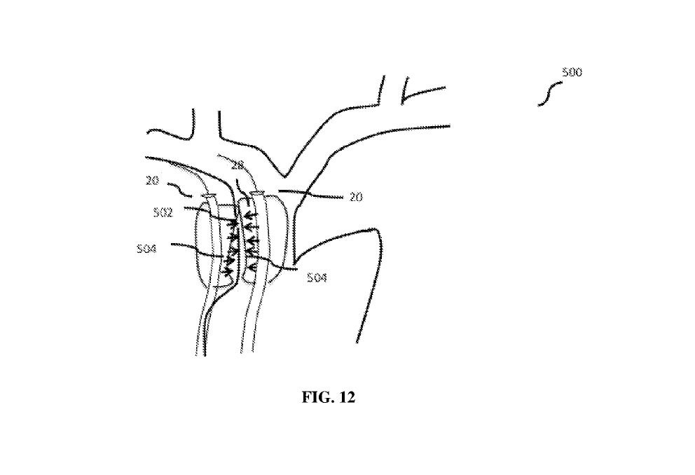

The catheter of the invention is used in some implementations to localize venous tears that sometimes occur during extraction procedures of chronic pacemaker or defibrillator leads. These leads can sometimes be adhered to the wall of the superior vena cava. During the extraction procedure, a tear in the SVC can sometimes result. The venous tears or holes can become a cause for trauma and excess bleeding and pose a great danger to the patient. Despite prompt surgical intervention, this condition has a very high mortality. By maneuvering the catheter at the appropriate site, the inflatable cuff can be used to plug the tears or holes that usually occur during the lead extraction, thus allowing sufficient time for healing, as well as in containing the extent of the tear by protecting the tear region through the seal function of the inflatable cuff of the catheter of the invention. FIG. 12 is a diagrammatic representation 500 (dual balloon approach) showing the deployment of two catheters 20 of the invention at a site of trauma having a vessel tear 502. The electromagnets (shown by arrows) 504 placed on the inflatable cuffs of the two catheters enable the sealing of the vessel tear. Thus, in another aspect of the invention, a method of vascular imaging using the catheter of the invention to locate sites of vessel trauma and sites of hemorrhage is described. The method includes steps for deploying the catheter adjacent to a vessel of interest using a guidewire; rotating the catheter such that the ventral surface of the inflatable cuff faces towards the site of possible vessel trauma; inflating the inflatable cuff to expand as a balloon and injecting dye to locate the site of trauma. The catheter with the inflated cuff is then maneuvered to envelop a desired location of the vessel of interest i.e. the site of trauma and hemorrhage where the inflatable cuff functions as a seal to prevent further bleeding into the pleural cavity or mediastinum.

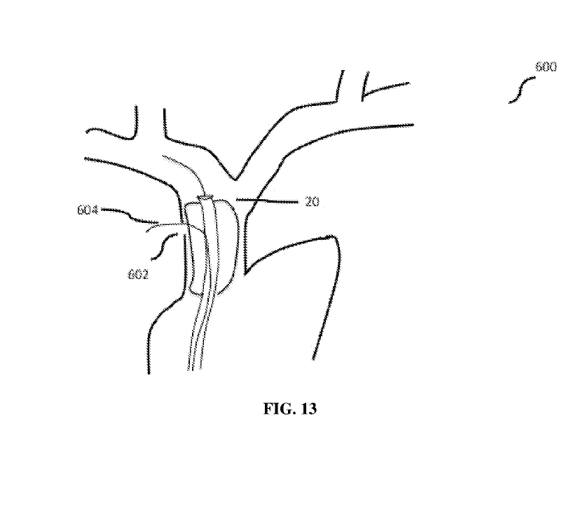

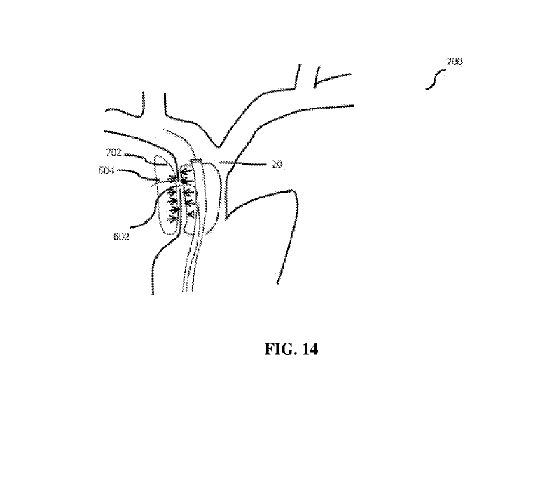

Also possible is the deployment through the catheter and over a wire of an expanding element on both sides of the vessel tear, to therefore plug the hole. FIG. 13 and FIG. 14 show the diagrammatic representation 600 and 700 respectively for such interventional procedures using the catheter 20 and the wire 604 at the site of trauma 602. Method for performing these interventional treatments using the catheter is also described herein. A wire 604 is passed through the catheter into the pleural cavity or other regions into where the hemorrhage is occurring as shown in FIG. 13 and FIG. 14. Following this step, over this wire, an expanding element 702 is deployed against the tear. This expanding element (that can consist of a sponge or a hydrogel or other materials) will have one component (first component) that will expand within the lumen of the vessel and another component (second component) that will expand outside the vessel, on the other side of the tear. The two components may be connected with a central waist and may also be drawn to each other with electromagnets as shown in FIG. 14 indicated by arrows. The expandable element in one example is a balloon that is pulled back to occlude the site of the tear.

Thus the different embodiments of the catheter disclosed herein are useful in both cardiac imaging and in interventional treatment procedures. Specifically these embodiments are useful in venography and radiological imaging of the right pericardiophrenic vein to detect and outline the course of the phrenic nerve, which is useful in planning any interventional treatment procedure in the vicinity of the phrenic nerve, and ensuring the safety for the phrenic nerve. The catheter may be similarly deployed in other cardiac regions where many anatomical structures overlap or crowd, and the inflatable cuff is deployed to provide a temporary seal for the target vessel or vessel of interest that lies in proximity or within the overlapping anatomical structures during the imaging or treatment procedure. This ensures that the contrast agent is injected into the proper vessel and does not overflow into the adjacent areas, as well as serves to protect the adjacent areas and the anatomical structures in those areas.

While only certain features of the invention have been illustrated and described herein, many modifications and changes will occur to those skilled in the art. It is, therefore, to be understood that the appended claims are intended to cover all such modifications and changes as fall within the true spirit of the invention.

We claim:

1. A catheter comprising:
a tube with one or more openings; and
an inflatable cuff holding a portion of the tube, wherein the inflatable cuff comprises a dorsal surface, and a ventral surface, wherein the ventral surface has a hollow compartment, an inner edge and an outer edge, and wherein the inflatable cuff functions as a seal to prevent an extrusion of an intravenous contrast agent supplied through one or more openings, in a dorsal direction.

2. The catheter of claim 1 wherein the inflatable cuff is an inflatable balloon.

3. The catheter of claim 1 wherein the inflatable cuff comprises a plurality of cuff openings for vacuum suction.

4. The catheter of claim 1 wherein the inflatable cuff is made of a material that is impermeable to the intravenous contrast agent.

5. The catheter of claim 1 wherein the inflatable cuff is made of a fabric material.

6. The catheter of claim 1 wherein the inflatable cuff is made of a polymer material.

7. The catheter of claim 1 wherein the outer edge and the inner edge comprise a compressible metallic frame.

8. The catheter of claim 1 wherein the compressible metallic frame is made from nitinol.

9. The catheter of claim 1 wherein the ventral surface comprising a plurality of magnets.

10. The catheter of claim 1 further comprising an ablation balloon disposed above the inflatable cuff, wherein the inflatable cuff is configured to expand as a shielding balloon.

11. A method of cardiac imaging comprising:
providing a catheter, wherein the catheter comprises a tube with one or more openings; and an inflatable cuff holding a portion of the tube, wherein the inflatable cuff comprises a dorsal surface, and a ventral surface, wherein the ventral surface has a hollow compartment, an inner edge and an outer edge, cuff openings on ventral surface;
deploying the catheter proximal to a vessel of interest;
rotating the catheter such that the ventral surface of the inflatable cuff faces ostium of the vessel of interest;
inflating the inflatable cuff to expand as a balloon to envelop the ostium of the vessel of interest;
deflecting the ventral surface of the catheter towards the ostium of the target vessel;
creating a seal between a wall of the vessel of interest and the inflatable cuff by applying suction forces through the cuff openings;
injecting an intravenous contrast agent into the vessel of interest through the one or more openings of the catheter; and
imaging the vessel of interest by detecting the intravenous contrast agent in the vessel of interest.

12. The method of claim 11 wherein deflecting comprises using electromagnets that are placed on the ventral surface of the inflatable cuff.

13. The method of claim 11 wherein deflecting comprises using pull wire mechanism for deflecting.

14. The method of claim 11 wherein the catheter is deployed using a guidewire, and wherein the inflatable cuff prevents the guidewire from going down the superior vena cava.

15. The method of claim 11 further comprising providing an ablation balloon for an interventional treatment procedure, wherein the inflatable cuff functions as a shielding balloon to protect an anatomical structure.

16. A method to locate a site of trauma in a vessel of interest, the method comprising:
deploying a catheter adjacent to the vessel of interest using a guidewire, wherein the catheter comprises a tube with one or more openings, and an inflatable cuff holding a portion of the tube, wherein the inflatable cuff comprises a dorsal surface, and a ventral surface, wherein the ventral surface has a hollow compartment, an inner edge and an outer edge, and wherein the inflatable cuff functions as a seal to prevent an extrusion of an intravenous contrast agent supplied through one or more openings, in a dorsal direction;
rotating the catheter such that the ventral surface of the inflatable cuff faces towards a site of possible vessel trauma in the vessel of interest;
inflating the inflatable cuff to expand as a balloon and injecting dye to locate the site of trauma; and
maneuvering the catheter to envelop the site of trauma where the inflatable cuff functions as a seal to prevent further bleeding.

17. A method for an interventional procedure at a site of trauma in a vessel of interest, the method comprising:
locating a site of trauma by deploying a catheter adjacent to the vessel of interest using a guidewire, wherein the catheter comprises a tube with one or more openings, and an inflatable cuff holding a portion of the tube, wherein the inflatable cuff comprises a dorsal surface, and a ventral surface, wherein the ventral surface has a hollow compartment, an inner edge and an outer edge, and wherein the inflatable cuff functions as a seal to prevent an extrusion of an intravenous contrast agent supplied through one or more openings, in a dorsal direction, and functions to envelop the site of trauma, where the inflatable cuff prevents further bleeding;
passing a wire through the catheter into the site of trauma;
deploying an expanding element through the wire, against a tear at the site of trauma, wherein the expanding element comprises a first component and a second component, wherein the first component expands within a lumen of the vessel of the vessel of interest and the second component expands outside the vessel of the vessel of interest, and wherein the first component and the second component are configured to connect.

18. The method of claim 17 wherein the expanding element is a hydrogel.

19. The method of claim 17 wherein the first component and the second component are configured to connect using a central waist.

20. The method of claim 17 wherein the first component and the second component are configured to connect using one or more electromagnets.
